(19) United States
(12) Patent Application Publication
(10) Pub. No.: US 2007/0239183 A1
(43) Pub. Date: Oct. 11, 2007

(54) Ophthalmic marker for surgical retinal vitreous

(76) Inventor: Toufic S. Melki, Rockville, MD (US)

Correspondence Address: JOHN A. RICHARDSON, 122 SUMMIT HALL ROAD, GAITHERSBURG, MD, 20877, US

(21) Appl. No.: 11/397,368

(22) Filed: Apr. 5, 2006

(51) Int. Cl. A61F 9/00

(52) U.S. Cl. 606/166

(57) ABSTRACT

The invention relates to opthalmology surgical procedures, and specifically to vitreous retinal surgical procedures for marking positions for incision/injection sites on the scleral surface of the eye. The types of ophthalmic vitreous surgical procedures for which are applicable include but not limited to, vitrectomies performed in hospital operating rooms and vitreous injections are routinely performed in doctor's offices for a variety of procedures to treat abnormal eye conditions. The invention discloses a range of scleral limbal marker for adult human patients, pediatric patients, and domestic and other animals. The invention discloses markers in materials capable of sterilization and for markers in materials suitable for disposal and packaged in vacuum sealed packages.

### BACKGROUND OF THE INVENTION

The invention herein disclosed describes an ophthalmic marker identified as the ‘Melki Marker’, and relates to opthalmology, and specifically to retinal surgical vitreous procedures for marking positions for incision/injection sites on the scleral tissue. The types of ophthalmic vitreous surgical procedures for which the ‘Melki Marker’ are applicable include but not limited to, vitrectomies performed in hospital operating rooms and in doctor's offices where vitreous injections are routinely performed for a variety of procedures to treat abnormal eye conditions.

In executing surgical complicated procedures on tissue within the human eye, and for other animals such as horses, domestic cats and dogs, it is necessary to accurately locate the position of incision locations on the scleral surface of the eye.

In order to prevent having surgical intervention taking place too far from the location to be operated on, or at a wrong angle or in an improper plane or inclination, it is necessary to mark the location as accurately and precisely as practical.

### BRIEF SUMMARY OF THE INVENTION

The specific procedure is an ophthalmic procedure wherein it is essential to locate the point of incision on the sclera limbal tissue surface to ensure that the location is safe to enter the Pars Plana region. In the event that the location is outside the Pars Plana region serious medical complications could result, such as lens damage, and thus creating a cataract. Another complication can be a retina tear or detachment. For the adult human eye the safe Pars Plana region is at a fixed dimension of 3.5 mm from the outer edge of the iris, also known as limbus.

The procedure using existing caliper markers consists of defining an incision site on the surface of the eyeball by initially drying the surface of the eye with sterilized disposable “Q” tip cotton swabs and marking the incision point with caliper pointers set by thumbscrew setting caliper scale at the required position to suit the patient. The set pointers are applied with ink by a nurse using sterilized blue pen from an epidermic labeler, such as #150 available from Devon Inc., or equivalent non-toxic, waterproof, absorbable ink or stain. In this manner the ink soaked pointers can be placed on the eye ball to mark the incision site.

It will be evident that in such a procedure, it is essential that the caliper pointers are correctly set by a nurse in attendance, further checked by the surgeon before carefully aligning caliper mechanism on the scleral in each instance and that the whole procedure is time consuming.

The marker types that have been used in these ophthalmic procedures are disclosed for example, by Simon in U.S. Pat. No. 5,090,955, and by manufacturer Storz Gmbh in Germany in the Model # E2404—Castroviejo Caliper. These markers are based on a caliper adjustment range of between 0 mm to 20 mm in 1 mm increments with a scale reading settings. The mode of operation is to use a thumbscrew setting to position the tips of the caliper pointers at the desired setting. In practice it is possible that errors result in the desired pointer-to-pointer setting due to haste in setting the pointers, wear and tear of the marker components and misreading of the scale reading setting. In addition, surgical tools of these types have non-smooth surfaces and hide-out areas that are required to be sterilized before reuse.

Simplicity is the essential feature of the Melki Marker invention in that it prevents the need for the surgeon to double-check the prior art ophthalmic markers caliper point settings that have been fixed by others. In addition, prior art caliper markers are subject to wear and tear of the setting mechanism that leads to caliper setting gauge inaccuracies after a period of time. By means of using a Melki Marker with a fixed pointer setting the surgeon saves time. The Melki Marker pointer setting is dependent on the type and age of the patient, for example, for an adult, the marker setting points are 3.5 mm apart and for premature infants and infants less than 3 months old, 1.0 mm apart, and for animals, such as racing horses, domestic pets, in the range 2.8 mm to 4.7 mm. These pointer settings are based on the need to ensure that the location of the incision point is in the safe Pars Plana region of the eye.

It is clear to one of ordinary skill in the art that there is a need for an error free, accurate and easy to use marker instrument that ensures the safety of ophthalmic procedures that require marking on the scleral surface of the eye.

In the preferred embodiment of the invention, the Melki Marker, the pointer set points are fixed for each category of patient whereby errors in pointer setting are eliminated and operating surgeon's time is reduced.

In the preferred embodiment the Melki Marker pointer settings are clearly identified on the instrument distel portion by engraving, imprinting or equal means, as a quality assurance feature to ensure that there is no possibility of using pointer settings in error.

In the preferred embodiment of the Melki Marker the material selected is one from a range of metal materials that are readily sterilizable and designed with smooth, shiny surfaces.

In a further preferred embodiment of the invention, the Melki Marker is made from materials of reinforced plastics, such as fiber glass, glass reinforced plastic or equal materials, that are less costly to make and can therefore be disposable after use.

In yet a further embodiment of the invention, the Melki Marker is arranged as a disposable once only use instrument having the pre-set pointer positions provided with pre-inked pointer tips with protector thimbles enclosing the ink-tips, and sealed in a sterilized see-through package, ready for immediate use by the operating surgeon pointer settings that are clearly identified on the instrument distel portion by engraving, imprinting or equal identification means.

### BRIEF DESCRIPTION OF THE DRAWINGS & TABLE

Various features, aspects, and advantages of the present invention will become more apparent with reference to the following Figures and Table accompanying this application, wherein:

FIG. 1—This Figure illustrates the fundamental eye elements for the purposes of performing vitreous retinal surgical procedures for marking incision/injection sites on the sclera limbal tissue.

FIG. 2—This Figure illustrates the marking on the scleral surface of the eye at a distance of 3.5 mm from the edge of the iris.

FIG. 3—This Figure shows vitrectomy instrument positions.

FIG. 4—This Figure shows an example of prior art used in scleral marking procedures.

FIG. 5—This Figure shows a further example of prior art used in scleral marking procedures.

FIG. 6—This Figure shows a practical example of the Melki Marker.

FIG. 7—This Figure shows image of Melki Marker with pointer distances for different patients.

FIG. 8—This Figure shows image of Melki Marker made in plastic or equal material with ink dipped pointers in sealed package.

TABLE 1—Depicts typical steps performed by a surgeon in marking the scleral of the human eye.

### LIST OF REFERENCE NUMBERS FOR THE ELEMENTS IN THE DRAWINGS

The following is a list of the elements in the drawings in numerical order.
1 Pupil
2 Iris
4 Vitreous
5 Retina
6 Sclera
10 Surgical Marker
12 Proximal End
14 Distel End
16 Bend Portion
18 Pointer
20 Pointer
22 Eyeball
24 Incision Spot
26 Caliper
28 Protector Thimbles for Pre-Inked dipped Pointers
30 Vacuum Sealed see-through Package

### DETAILED DESCRIPTION OF THE INVENTION

The object of the present invention is therefore to provide a marker for ophthalmic surgical procedures which permits the operating physician to effect a precise marking of the location to be operated upon with accuracy, safety and a reduction in the surgeon's operating time.

For such procedures, the Melki Marker is held by the surgeon in one hand and brought into contact with the sclera limbal tissue of the eye by means of the operating physician taking hold of a tubular marker at the distel handle end and presenting the two sharp marker pointers at the proximal end so that two dots may be made on the scleral surface at for example, 3.5 mm apart and in a manner that uses the edge of the iris, also known as limbus, as reference point and ensures that the Pars Plana safety region is maintained. The novel feature of the ‘Melki Marker’ is that by having a fixed distance between the marker points it is ensured that incision spots 24 made by the pointers are always 3.5 mm apart for adult human procedures.

The key embodiment of the Melki Marker is that it is a simple and easy to use surgical instrument that has no moving parts that are intrinsic to prior art caliper markers. The absence of moving parts prevents marker manipulation position settings errors, errors in settings due to wear and tear of the caliper mechanism moving parts, and saves the surgeon the need to double-check the caliper setting for inaccuracies.

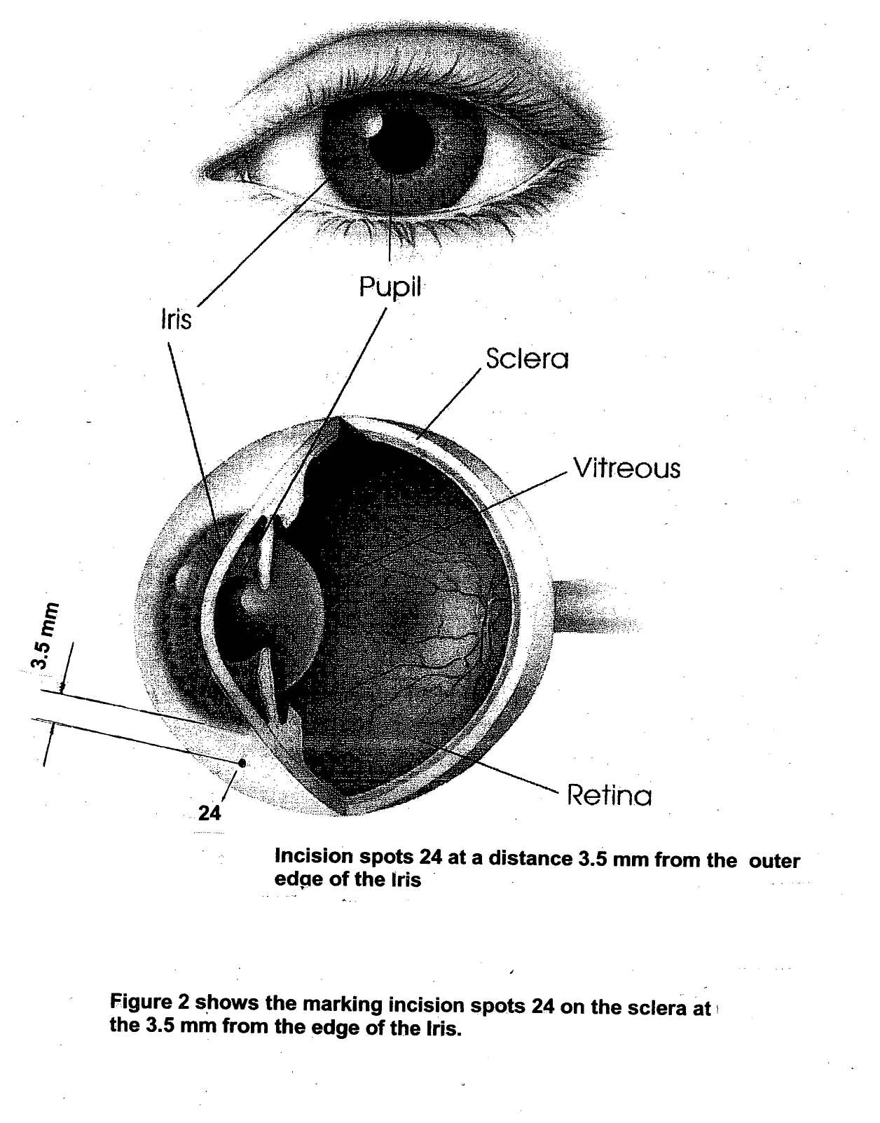

There are several ophthalmic procedures that require the surgeon to locate precise incision points on the scleral surface of the eye. For example, for vitrectomies performed in hospital operating rooms, and in out-patient surgical facilities, it is necessary to locate up to three incision spots 24 on the scleral surface wherein each location is required to be in the Pars Plana safety region at exactly 3.5 mm from the edge of the iris as shown in FIG. 2.

The sharp marker pointer ends 12 and 18 are applied with ink by a nurse using sterilized blue ink pen from an epidermic labeler, such as #150 available from Devon Inc., or equivalent non-toxic, waterproof, absorbable ink or stain. In this manner the ink soaked pointers can be placed on the eye ball to mark multiple incision/injection spots 24 on scleral surface in such Operating Room settings where for example, vitrectomy ophthalmic procedures are performed.

There are other ophthalmic surgical procedures that are performed in doctor's offices where the treatment requires the injection of medications such as steroids, anti-biotics, and anti-VEGF and requires similar procedures/injections for the location of injection spots 24.

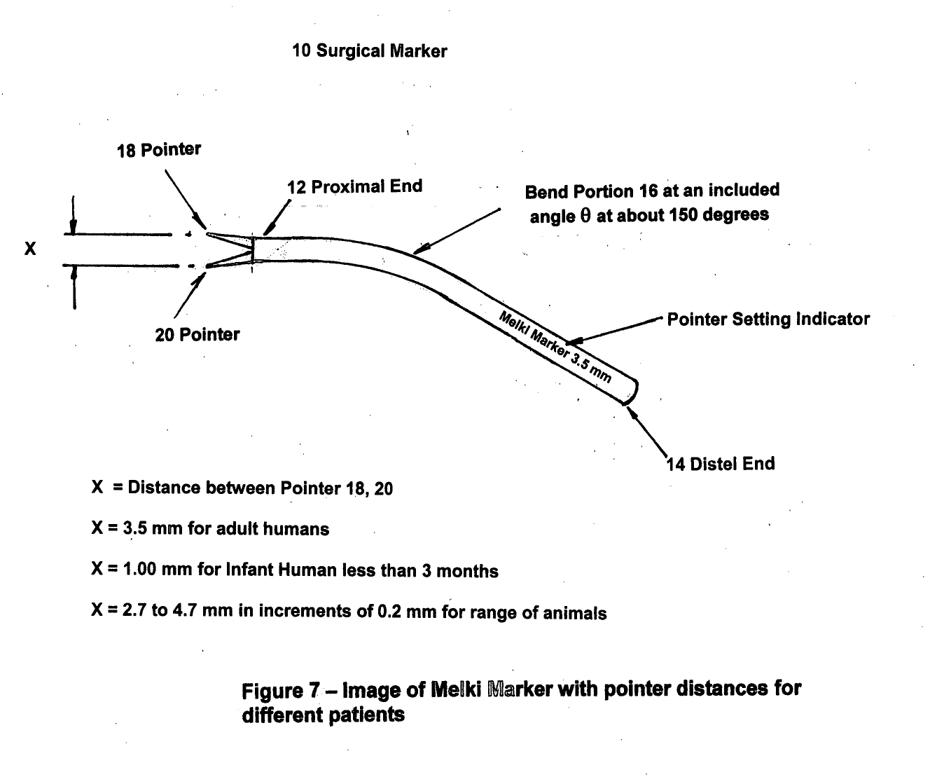

In a preferred embodiment of the surgical marker the Melki Marker is made from stainless steel or equal material in the form of a solid smooth surface tubular metal rod and arranged with the marker points at exactly 3.5 mm apart for use on adult human eyes as disclosed in FIG. 7. This embodiment can be provided with an identification marker (such as “Melki Marker 3.5 mm, or “M-M 3.5 mm”) on the handle of the instrument to indicate the pointer setting distance in mm's and this can be applied by engraving, imprinting, stamping, stenciling or other equal identification method.

In a further preferred embodiment the Melki Marker is made from reinforced plastics, such as fiber glass, glass reinforced plastic or equal light weight, high tensile strength materials in the form of a solid smooth surface tubular rod, that is less costly to make than metallic forms and can be disposable after use. This embodiment can also be provided with an identification marker on the handle of the instrument to indicate the pointer setting in mm and this can be applied by engraving, imprinting, stamping, and stenciling or other equal identification method.

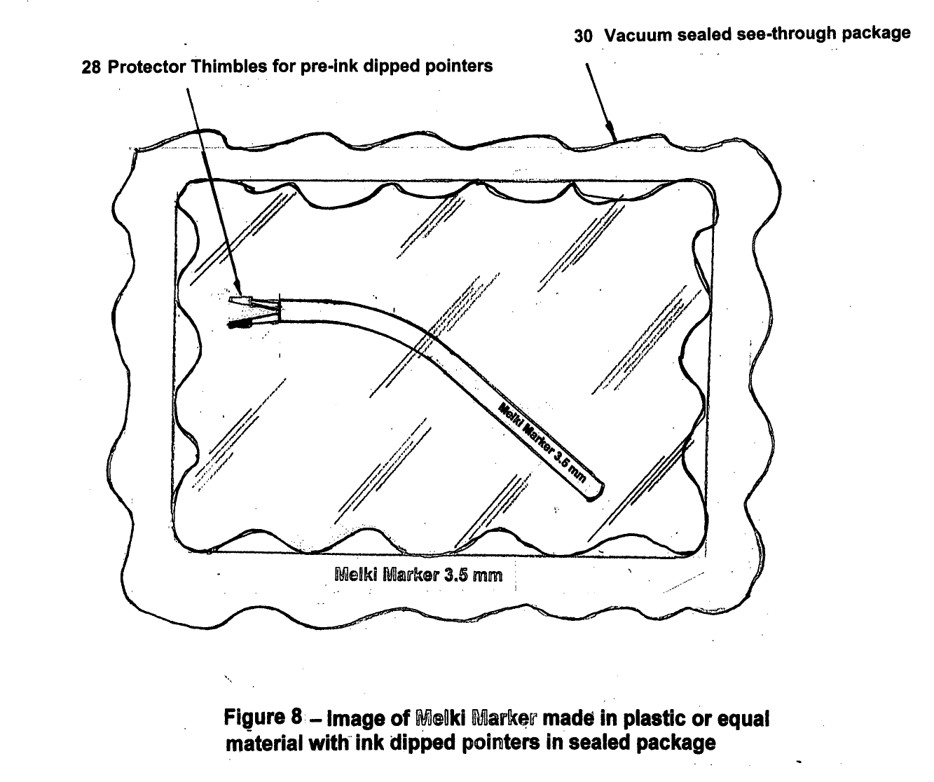

In yet a further preferred embodiment the distel ends of the sharp pointers 18, 20, markers are pre-soaked in ink dye from an approved epidermic labeler and protected by rubber, polystyrene or other equal type of material thimbles 28, and packaged for use in a sterilized sealed see-through package 30, as disclosed in FIG. 8. In this embodiment the material selection makes it suitable for disposal after use. In addition, in this embodiment the handle portion of the instrument is engraved, stamped, imprinted or other identifying means, with the pointer distances in mm. In addition, the see-through sealed package can also be imprinted, stamped, or with equal identification means to indicate to the surgeon the marker pointer distance in mm's.

FIG. 7 shows a specific embodiment of a surgical marker made from high tensile strength material, such as stainless steel, alloy steel, titanium or equal material that can be sterilized. The surgical marker 10 has a tubular distel end 14 with a means for readily handling and a bend portion 16 at an included angle of approximately 150 degrees for an ergonomic configuration, a proximal end 12 with two sharp-ended pointers 18 and 20 arranged in a vee formation and set at a fixed distance of 3.5 mm between said pointers 18, 20 in a preferred embodiment for adult patients.

The invention described herein is for a surgical instrument used primarily intended for surgical retinal surgeons. However, the basic concept of the invention could have other applications in other medical specialties with different proximal end pointer settings in excess of 5.00 mm.

Table 1 shows typical steps performed by the physician for scribing the eye sclera incision/injection site(s).

The Melki Marker invention incorporates a number of alternative embodiments for the material for making the marker. For example, a preferred embodiment of the surgical marker the material of the marker can be made of alloy steels, stainless steel, nickel-chromium steels, or titanium that can be readily sterilized in the hospital or doctor's office facilities. A further preferred embodiment of the invention incorporates a disposable material, such as fiber-glass, glass reinforced plastic (GRP) or equal light weight high strength materials, for the Melki Marker wherein the marker can be disposed in licensed disposal equipment.

The above developments of the marker tool are indicative of use in medical procedures wherein the marker can be used in both hospital and doctor's office settings. It will be clear from the foregoing specification disclosure and drawings, that the invention of the Melki Marker provides a simple and reliable means for marking incision/injection spots on the scleral libel tissues of the human and other animal eye surfaces that is easy to use, exactly accurate, not prone to operating room setting errors, and not subject to inaccuracies due to wear and tear of existing caliper marker setting appliances.

The preferred Melki Marker embodiment is made of materials such as stainless steels, alloy steels, titanium or equal high tensile strength materials that provide for instrument re-use after sterilization, is particularly suitable for hospital operating room settings, as disclosed for example, in FIG. 7.

In a further preferred embodiment the Melki Marker made out of low cost, light weight, high tensile strength plastic materials, and disposable after use is suitable for doctor's office settings. This particular embodiment in the form of a pre-packaged instrument as disclosed in FIG. 8 is ideal for use in doctors' offices.

While the invention has been described in connection with what is presently considered to be the most practical and preferred embodiments, it is understood that the invention is not limited to the foregoing Detailed Description Drawings of the Invention, and disclosed preferred embodiments, but it will be appreciated by those skilled in the art that the Invention is not limited to the embodiments disclosed, but is capable of numerous rearrangements, modifications, and substitutions of parts or elements without departing from the spirit and scope of the appended Claims.

### DESCRIPTION OF TABLE

TABLE 1—Depicts typical steps performed by a surgeon in marking eye scleral surface.

TABLE 1

| STEP NUMBER | DESCRIPTION |
|---|---|
| STEP 1 | Prepare the patient by administering eye drops/ |
| | medication for dilation, sedation, anesthesia |
| STEP 2 | Prepare surface of the sclera conjunctive tissue |
| | of the eye by drying with sterilized “Q” |
| | tips cotton swabs |
| STEP 3 | Select specific Melki Marker with indicator |
| | confirming that the pointers are at fixed |
| | distances apart to suit patient |
| Alternative | Select specific disposable Melki Marker in vacuum |
| STEP 3 | sealed see-through package with pre-inked pointer |
| | tips |
| STEP 4 | Soak the pointers by application of approved |
| | disposable blue ink pen |
| STEP 5 | Present ink-tipped pointers to the surface of |
| | the scleral surface to make incision/injection |
| | mark(s) within the Pars Plana safety region |

### DESCRIPTION OF FIGURES

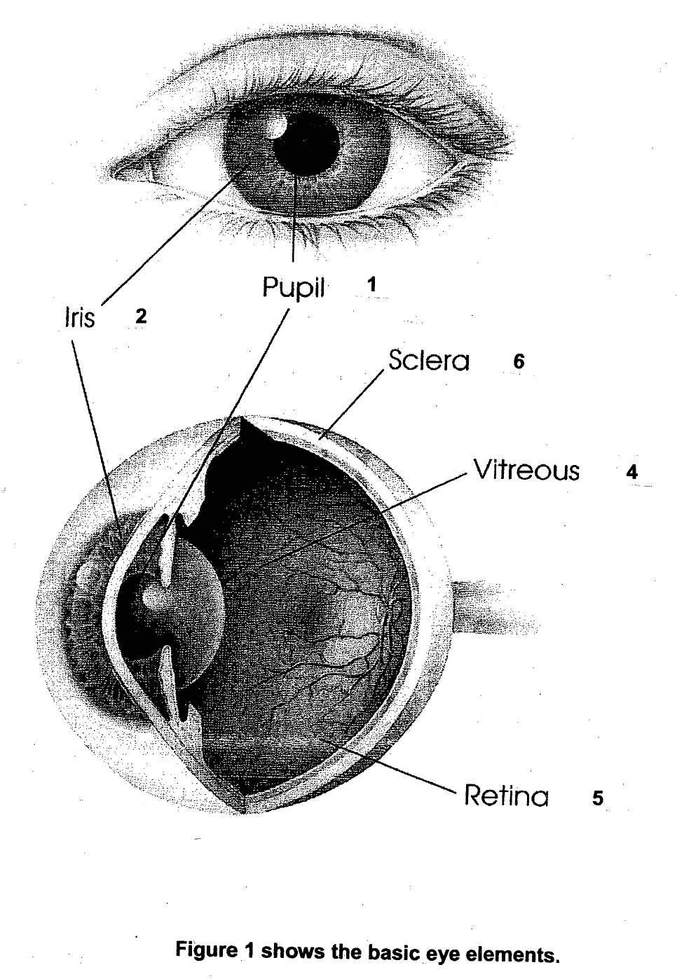

FIG. 1—This Figure illustrates the fundamental eye elements for the purposes of performing vitreous retinal surgical procedures for marking incision sites on the sclera limbal tissue.

FIG. 2—This Figure illustrates the marking on the scleral surface of the eye at a distance of 3.5 mm from the edge of the iris.

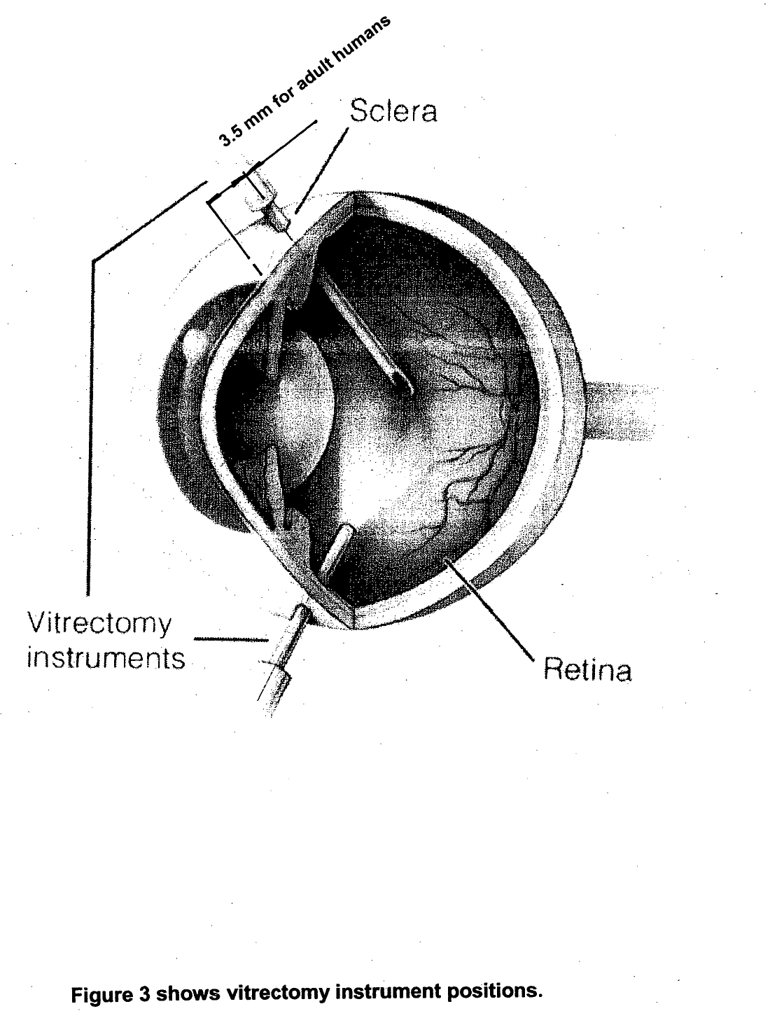

FIG. 3—This Figure shows vitrectomy instrument positions.

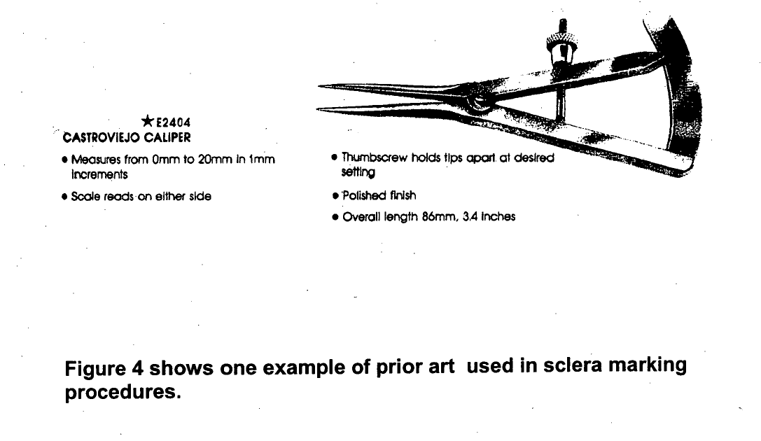

FIG. 4—This Figure shows an example of prior art used in scleral marking procedures.

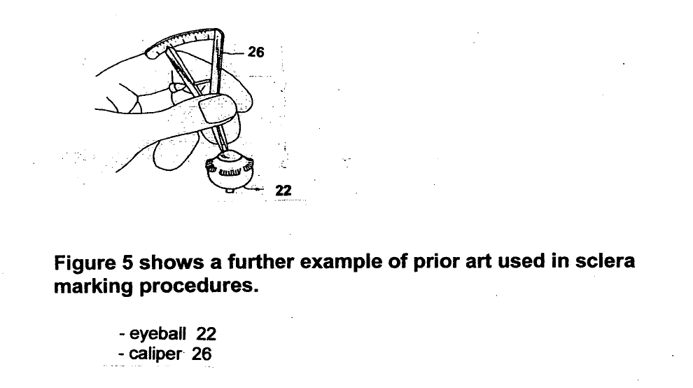

FIG. 5—This Figure shows a further example of prior art used in scleral marking procedures.

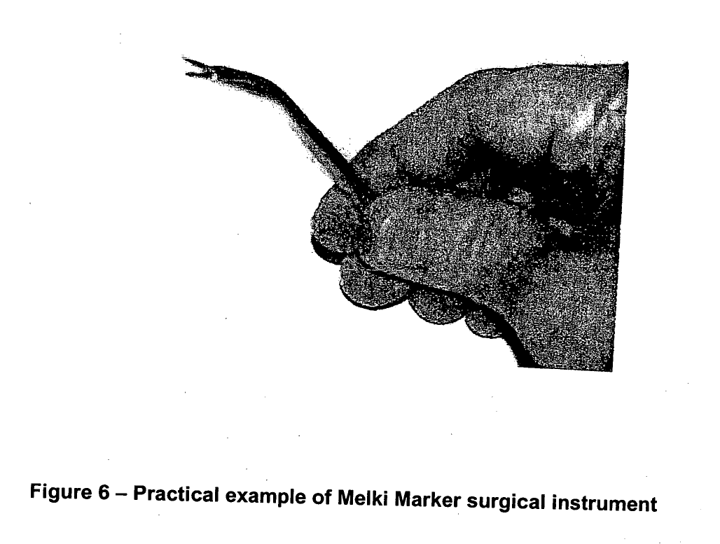

FIG. 6—This Figure shows a practical example of the Melki Marker.

FIG. 7—This Figure shows the image of the Melki Marker with pointer distances for different patients.

FIG. 8—This Figure is a further preferred embodiment of the Melki Marker invention using plastic or equal material and wherein the end-pointers are soaked in FDA approved ink, protected by thimbles and the vacuum sealed in see-through package.

I claim:

1. A surgical instrument for marking locations on the surface scleral of the eye for selecting injection or incision sites, comprising:
a) an elongated solid tubular structure defining a distel end adapted to being held by a surgeon and with a bend section at approximately at mid-point of the said structure in an ergonomic configuration, and a proximal end incorporating two sharp end pointers for marking purposes arranged in a vee formation and set at a fixed distances apart in the range of 1.00 mm to 5.00 mm, and,
b) whereby the said instrument is presented by said surgeon to the said scleral surface and whereby from the said distel end said surgeon is able to accurately scribe an incision spot at an exact position based on the said fixed distance between the said sharp pointers from the edge of the eye iris also known as limbus to ensure that the incision is within safety region known as Pars Plana.

2. The surgical instrument of claim 1 wherein the said elongated solid tubular structure is made from stainless steel, alloy steel, titanium or equal high tensile strength metallic material.

3. The surgical instrument of claim 1 wherein the said elongated solid tubular structure is made from fiber glass, glass reinforced plastic or other equal light weight and high tensile strength plastic material.

4. The instrument of claim 1 wherein the said fixed distance between the sharp end pointers is 3.5 mm for adult patients.

5. The instrument of claim 1 wherein the said fixed distance between the sharp end pointers is 1.0 mm apart for pediatric premature infants and infants less than 3 months old.

6. The instrument of claim 1 wherein the said fixed distance between the sharp end pointers are set at fixed distances for animals, such as race-horses, dogs, cats, in the range of 2.7 mm to 4.7 mm at about 0.2 mm increments.

7. The instrument of claim 3 wherein the said instrument is packaged in a sterilized vacuum sealed see-through package.

8. The instrument of claim 7 wherein the said instrument has the pointers soaked inappropriate FDA approved ink dye and protected by thimbles and enclosed in said sterilized vacuum sealed see-through package.

9. A method for creating a marking on the surface of the scleral of the eye, comprising the steps of:
a) prepare the patient by administering eye drops for dilation and sedation purposes and,
b) prepare the surface of the said eye scleral by drying the surface of the eye with sterilized disposable “Q” tip cotton swabs,
c) select specific marker with pointers at specific fixed distance apart to suit the patient and,
d) soak pointers using a sterilized blue pen and,
e) use soaked pointers of the selected marker to make prescribed incision or injection spots from the edge of the iris also known as limbus and within a safety region known as Pars Plana of the sclera surface thereby defining a site for surgical injection or incision.

10. The method of claim 9 whereby the method is performed in a hospital setting to make multiple incision or injection spots for use in vitrectomy ophthalmic procedures.

11. The method of claim 9 whereby the method is performed in a doctor's office to inject medications.

12. A surgical instrument for marking locations on the scleral surface of the eye for selecting injection or incision sites, comprising:
a) an elongated solid smooth surface tubular structure manufactured from materials, such as stainless steel, titanium or equal high tensile strength material, and,
b) said elongated structure defining a distel end adapted to being held by a surgeon and with a bend section at approximately at mid-point of the said structure in an ergonomic configuration, and with a proximal end incorporating two sharp end pointers for marking purposes arranged in a vee formation and set at a fixed distance of 3.5 mm apart, and,
c) whereby the said instrument is presented to the said scleral surface by said surgeon to accurately scribe an incision spot at an exact position based on the said fixed distance between the said sharp pointers from the edge of the eye iris also known as limbus to ensure that the incision is within a safety region of the eye known as Pars Plana.

13. The surgical instrument of claim 12 is made from fiber glass, glass reinforced plastic or other equal light weight and high tensile strength plastic material.
